(19) United States
(12) Patent Application Publication
(10) Pub. No.: US 2008/0139464 A1
(43) Pub. Date: Jun. 12, 2008

(54) Isolated Ny-Eso-1 Peptides Which Bind To Hla Class II Molecules And Uses Thereof

(75) Inventors: Sacha Gnjatic, New York, NY (US); Djordje Atanackovic, Hamburg (DE); Lloyd J. Old, New York, NY (US)

Correspondence Address: Norman D Hanson;Fulbright & Jaworski, 666 Fifth Avenue, New York, NY, 10103, US

(73) Assignees: LUDWIG INSTITUTE OF CANCER RESEARCH, New York, NY (US); MEMORIAL SLOAN-KETTERING CANCER CENTER, New York, NY (US)

(21) Appl. No.: 10/556,853

(22) PCT Filed: May 27, 2004
(86) PCT No.: PCT/US2004/016849

§ 371 Date: Feb. 11, 2008

Related U.S. Application Data
(60) Provisional application No. 60/474,893, filed on May 30, 2003.

(51) Int. Cl. A61K 38/17; C07K 14/47; A61K 38/08; C12N 15/85; A61P 43/00; C12N 5/10; C12N 15/12; C07K 7/00

(52) U.S. Cl. 514/12; 530/324; 530/327; 514/15; 536/23.5; 435/325; 435/320.1

(57) ABSTRACT

The invention relates to peptides which consist of amino acid sequences found in the NY-ESO-1 molecule, which bind to MHC-Class II molecules. These can be used alone, or in combination with other peptides.

### FIELD OF THE INVENTION

This invention relates to HLA binding peptides derived from an antigen associated with cancer. These peptides bind to Class II MHC molecules.

### BACKGROUND AND PRIOR ART

It is fairly well established that many pathological conditions, such as infections, cancer, autoimmune disorders, etc., are characterized by the inappropriate expression of certain molecules. These molecules thus serve as “markers” for a particular pathological or abnormal condition. Apart from their use as diagnostic “targets”, i.e., materials to be identified to diagnose these abnormal conditions, the molecules serve as reagents which can be used to generate diagnostic and/or therapeutic agents. A by no means limiting example is the use of a peptide which complexes with an MHC molecule, to generate cytolytic T cells against abnormal cells.

Preparation of Such Materials, of Course, Presupposes a Source of the Reagents Used to generate these. Purification from cells is one laborious, far from sure method of doing so. Another preferred method is the isolation of nucleic acid molecule which encode a particular marker, followed by the use of the isolated encoding molecule to express the desired molecule.

To date, two strategists have been employed for the detection of such antigens, in e.g., human tumors. These will be referred to as the genetic approach and the biochemical approach. The genetic approach is exemplified by, e.g., dePlaen et al., Proc. Natl. Sci. USA 85:2275 (1988), incorporated by reference. In this approach, several hundred pools of plasmids of a cDNA library obtained from a tumor are transfected into recipient cells, such as COS cells, or into antigen-negative variants of tumor cell lines which are tested for the expression of the specific antigen. The biochemical approach, exemplified by, e.g., O. Mandelboim, et al., Nature 369:69 (1994), incorporated by reference, is based on acidic elution of peptides which have bound to MHC-Class I molecules of tumor cells, followed by reversed-phase high performance liquid chromography (HPLC). Antigenic peptides are identified after they bind to empty MHC-Class I molecules of mutant cell lines, defective in antigen processing, and induce specific reactions with cytotoxic T-lymphocytes. These reactions include induction of CTL proliferation, TNF release, and lysis of target cells, measurable in an MTT assay, or a ⁵¹Cr release assay.

The two approaches to the molecular definition of antigens described supra have the following disadvantages: first, they are enormously cumbersome, time-consuming and expensive; and second, they depend on the establishment of cytotoxic T cell lines (CTLs) with predefined specificity.

The problems inherent to the two known approaches for the identification and molecular definition of antigens is best demonstrated by the fact that both methods have, so far, succeeded in defining only very few new antigens in human tumors. See, e.g., van der Bruggen, et al., Science 254:1643-1647 (1991); Brichard, et al., J. Exp. Med. 178:489-495 (1993); Coulie, et al., J. Exp. Med. 180:35-42 (1994); Kawakami, et al., Proc. Natl. Acad. Sci. USA 91:3515-3519 (1994).

Further, the methodologies described rely on the availability of established, permanent cell lines of the cancer type under construction. It is very difficult to establish cell lines from certain cancer types, as is shown by, e.g., Oettgen, et al., Immunol. Allerg. Clin. North. Am. 10:607-637 (1990). It is also known that some epithelial cell type cancers are poorly susceptible to CTLs in vitro, precluding routine analysis. These problems have stimulated the art to develop additional methodologies for identifying cancer associated antigens.

One additional methodology is described by Sahin, et al., Proc. Natl. Acad. Sci. USA 92:11810-11913 (1995), incorporated by reference. Also, see U.S. Pat. No. 5,698,396, incorporated by reference. To summarize, the method involves the expression of cDNA libraries in a prokaryotic host. (The libraries are secured from a tumor sample). The expressed libraries are then immunoscreened with absorbed and diluted sera, in order to detect those antigens which elicit high titer humoral response. This methodology is known as the SEREX method (“Serological identification of antigens by Recombinant Expression Cloning”). The methodology has been employed to confirm expression of previously identified tumor associated antigens, as well as to detect new ones. See the above referenced patent applications and Sahin, et al., supra, as well as Crew, et al., EMBO J. 144:2333-2340 (1995).

One important antigen identified by the SEREX methodology is referred to as NY-ESO-1. The antigen is described in, e.g., U.S. Pat. No. 5,804,381, and Chen, et al., Proc. Natl. Acad. Sci. USA 94:1914-1918 (1997), the disclosures of which are incorporated by reference. Originally, NY-ESO-1 was characterized as an antigen which was processed into peptides presented by MHC Class I molecules. Later work showed that it also processed into peptides that are presented by MHC Class II molecules. See Jager, et al., J. Exp. Med. 191:625 (2000), as well as PCT application publication number WO99/53938, published Oct. 28, 1999, both of which are incorporated by reference in their entirety. Also, see WO 01/23560, also incorporated by reference. Additional papers have been published which describe additional peptides which consist of amino acid sequences found in NY-ESO-1, which also bind to MHC Class II molecules and serve as T cell epitopes. Exemplary are Zeng, et al., J. Immunol 165:1153-1159 (2000); Zarour, et al., Canc. Res. 60:4946-4952 (2000); Zarour, et al., Canc. Res. 62:213-218 (2002); Zeng, et al., Proc. Natl. Acad. Sci. USA 98(7):3964-3969 (2001), and Zeng, et al., Canc. Res. 62:3630-3635 (2002), all of which are incorporated by reference. It has also been shown that humoral responses to NY-ESO-1 correlate with the presence of peripheral CD8+ T cells against NY-ESO-1. See Jager, et al., Proc. Natl. Acad. Sci. USA 97:4760-4765 (2000).

The interest in such molecules results from several factors. First, NY-ESO-1 appears to be restricted in its expression to tumor cells, of various histological types, and male germ cell lines. Exemplary of the tumor types in which NY-ESO-1 expression is found are melanoma, breast, prostate, lung, urinary bladder, carcinoma, and synovial sarcoma. See Jäger, et al., supra. Also see Chen, et al., supra Stockert, et al., J. Exp. Med. 187:1349 (1998); Wang, et al., J. Immunol. 161:3598-3606 (1998); Jungbluth, et al., Int. J. Cancer 92:856-860 (2001); Jungbluth, et al., Int. J. Cancer 94:252-256 (2001); all incorporated by reference.

The fact that T cells play an important role in controlling tumor growth and mediating tumor regression is well known. The molecular mechanisms underlying T cell mediated anti-tumor immunity has been elucidated, inter alia by the identification of tumor antigens that are recognized by CD8⁺ T cells. See Rosenberg, Immunity 10:281-287 (1998); Wang, et al., Immunol. Rev. 170:85-100 (1999). The advances in the identification of such molecules have led to their use in clinical trials, examples of which may be seen in Rosenberg, Nature 411:380-384 (2001). Also see Nestle, et al., Nat. Med. 4:328-332 (1998); Rosenberg, et al., Nat. Med. 4:321-327 (1998); Lee, et al., J. Clin. Oncol. 19:3836-3847 (2001); Thurner, et al., J. Exp. Med. 190:1669-1678 (1999).

The growing interest in MHC Class II presentation stems, in part, from animal model studies that indicate that it may be necessary to engage CD4⁺ T cells as well as CD8⁺ T cells in order to develop effective cancer vaccines. See Zeng, J. Immunother 24:195-204 (2001).

To move from the general back to the specific, NY-ESO-1, as has been pointed out, supra, shows strict tumor expression. In addition to the CD8+ T cell response noted supra, high titers of NY-ESO-1 antibodies were present in patients who express the molecule, suggesting that there is a CD4⁺ T cell response involved. See Wang, et al., Immunol. Rev. 179:85-100 (1999); Pardoll, et al., Curr. Opin. Immunol 10:588-594 (1998); and Jager et al., Proc. Natl. Acad. Sci. USA 97:4760-4765 (2000). While NY-ESO-1 derived peptides (“derived” as used herein, refers to contiguous amino acid sequences which can be found in the NY-ESO-1 protein sequence described in the Chen '381 patent and PNAS paper cited supra) have been identified which are presented by HLA-DRB1*0401 and HLA-DRB1*0101 (Zeng, et al., J. Immunol 165:1153-1159 (2000); Jager, et al., J. Exp. Med. 191:625-630 (2000)), the majority of patients who present NY-ESO-1 specific antibodies do not express these MHC-Class II molecules. Hence, there is an interest in finding additional peptides, derived from NY-ESO-1, which bind to MHC-Class II molecules, for all of the reasons described supra. Further, there is a need to extend the use of peptide vaccines to patients who present other types of MHC Class II molecules.

It is not clear why an intracellular antigen like NY-ESO-1 is capable of eliciting antibody responses in cancer patients. The hypothesis is that when a tumor reaches a critical mass, necrotic areas start releasing protein or polypeptide antigens that become accessible to APCs that in turn can prime coordinated B cell and T cell responses. The idea that CD4⁺ T cells are involved early on may explain why antibodies to NY-ESO-1 are of IgG isotypes rather than IgM. See Stockert, et al., J. Exp. Med. 187: 1349-1354 (1998). Helper T cells were shown to be critical for inducing antibody class switching by B cells. The major pathway for class switching occurs through surface interaction/stimulation of CD40 with CD40L (Oxenius, et al., J. Exp. Med. 183:2209-2218 (1996); Dullforce, et al., Nat. Med. 4:88-91 (1998)), and with additional involvement of more recently described molecule ICOS. See McAdam, et al., Nature 409:102-105 (2001).

Independently from their helper role in humoral responses, CD4⁺ T cells also provide a protective function by cytokine secretion and local inflammatory reactions, which has been shown to be independent of the CD40-CD40L interaction. See Oxenius, et al., J. Exp. Med. 183:2209-2218 (1996). It is thus interesting to determine if CD4⁺ T cell responses to NY-ESO-1 are dependent on the presence of antibody to NY-ESO-1, as are CD8+ T cell responses, or whether CD4⁺ T cell responses to NY-ESO-1 could be seen more broadly in patients, in a first line of immunity preceding the other actors of the adaptive response.

CD4⁺ T cell responses to NY-ESO-1 have been recently described. Large epitopes restricted by HLA-DR4 (Jager, et al., J. Exp. Med. 191:625-630 (2000); Zarour, et al., Cancer Res. 60:4946-4952 (2000)) or promiscuous for several HLA-DR have been shown. See Zarour, et al., Cancer Res. 62: 213-218 (2002). BLA-DP4-restricted responses were also seen and were suggested to correlate with antibody status. See Zeng, et al., Proc. Natl. Acad. Sci. USA 98:3964-3969 (2001). Still, most approaches to study CD4⁺ T cells rely on the generation of T cell lines following multiple peptide stimulations, assessed for bulk cytokine secretion or proliferation. The lack of reliable and quantitative approaches for monitoring CD4⁺ T cell responses has hindered the analysis of larger series of patients.

There are various “rules” or “guidelines” for determining if a peptide of interest should bind to a given MHC or HLA molecule. See, for example, Marsh, et al., The HLA Factsbook (Academic Press, 2000), which presents a listing of “binding motifs” for determining the likelihood of a peptide binding to a particular MHC Class I or Class II molecule. There are also numerous algorithms and programs available to facilitate this review. See, e.g., Southwood, et al., J Immunol 160:3363 (1998); Honeyman, et al., Nat. Biotechnol. 16:966-969 (1998); Breisie, et al., Bioinformatics 14:121-131 (1998), as well as the “SYFPEITHI” algorithm, referred to infra. As will be shown, experimental conformation of these algorithms is always required before any conclusions can be drawn. The common occurrence of false positives is a major drawback of algorithm defined, HLA binding peptides.

Responses of purified CD4⁺ T cells to the model antigen influenza nucleoprotein (NP) were assessed after a single round of in vitro antigen stimulation, to recall memory responses only. Specific CD4⁺ T cells were detected not only against a peptide containing HLA-DR promiscuous epitope, but also against full-length NP expressed in APCs from recombinant fowlpox vectors. The use of innovative targets, namely PHA-expanded CD4⁺ T cells (T-APC), meant that ELISPOT assays could be carried out to measure specific T cell responses with virtually no background, in a quantitative and reproducible manner.

The disclosure which follows identifies new Class II binding peptides derived from NY-ESO-1. The ramifications of this discovery are also a part of this invention, as will be seen from the disclosure which follows. The methodology employed is the same approach that was described supra, i.e., NY-ESO-1 specific CD4⁺ T cell response can be monitored in cancer patients via analysis of NY-ESO-1 expression in tumor cells and patient seropositivity to NY-ESO-1. The approach which is not limited to known epitopes, is applicable to any patient, regardless of HLA restriction. As will be seen from the disclosure which follows, new HLA Class II restricted peptides can be identified in this way.

### EXAMPLE 1

In these experiments, cells were used which originated from a patient who had been shown to have a spontaneous, CD4⁺ T cell response to certain NY-ESO-1 derived peptides, i.e., peptides consisting of amino acids 115-132, 121-138, and 139-156 of the NY-ESO-1 amino acid sequence, referred to supra.

First, peripheral blood mononuclear cells (“PBMCs” hereafter) were obtained. Then, CD4⁺ and CD8⁺ T cells were separated therefrom, using antibody coated magnetic beads and standard methods.

Once the cells were separated, the CD4⁺ T cells were seeded into round bottomed 96 well plates, at a concentration of 5×10⁵ cells/well, in RPMI medium 1640 supplemented with 10% human AB serum, L-glutomine (2 mM) penicillin (100 U/ml), strepto-mycin (100 μg/ml), and 1% nonessential medium.

The CD4⁺ T cells were sensitized by antigen presenting cells (“APCs”), which were prepared from autologous PBMCs which had CD4⁺ and CD8⁺ cells removed. These depleted PBMCs were either pulsed with NY-ESO-1 derived peptides (10 μm), or infected with recombinant adenovirus encoding full length NY-ESO-1, at 1000 infectious unit/cell, prepared in accordance with Gnjatic, et al., Proc. Natl. Acad. Sci. USA, 97:10917-10922 (2000), incorporated by reference, overnight at 37° C. in 250 μl serum free medium. Either the pulsed, or infected APCs were then washed, irradiated, and added to the CD4⁺ T cells, at concentrations of 1×10⁶ APCs/well. After 8 hours, IL-2 (10 U/ml), and IL-7 (20 ng/ml) were added, and then added again every 3-4 hours.

This resulted in presensitized T cells, which were tested in ELISPOT assays, as described in the experiment which follows.

### EXAMPLE 2

In the ELISPOT assay, mAb against IFN-γ was used to coat flat bottomed, 96-well nitrocellular plates (2 μg/ml), and incubated at 4° C., overnight. The plates were washed with RPMI and blocked with 10% human AB serum for 2 hours, at 37° C.

Then, 5×10⁴ presensitized CD4⁺ T cells were added, together with 1×10⁵ target cells. The target cells were either autologous activated T cell APCs (T-APCs), or Epstein Barr Virus (EBV) transformed B cells. The T-APCs were prepared by taking a portion of the CD4⁺ T cells separated as described, supra, seeding them into 48 well plates, at a concentration of 1×10⁶ cells/ml in complete medium, supplemented with 10 μg/ml PHA. Cells were fed and expanded twice a week, using complete medium with IL-2 (10 U/ml), and IL-7 (20 ng/ml). These T-APCs were used after about 20 days of culture. These were pulsed as described, supra.

EBV transformed B cells were cultured in RPMI medium 1640 that had been supplemented with 10% FCS, L-glutamine (2 ml) penicillin (100 U/ml), streptomycin (100 μg/ml), and 1% nonessential amino acids.

After adding the target cells, the mixtures were incubated for 20 hours in serum free, RPMI medium. Plates were washed thoroughly with water containing 0.05% Tween to remove cells, and 0.2 μg/ml of anti-IFN-γ monoclonal antibodies, labeled with biotin, were added. These were then developed with streptavidin/alkaline phosphatase conjugates (1 μg/ml), for 1 hour at room temperature. These were washed, and 5-bromo-4-chloro-3-indolyl phosphate/nitroblue tetrazolium was added and incubated for 5 minutes.

Final washing followed and then any plate membranes which displayed dark violet spots were scanned and the number of spots was counted.

In control experiments used in the development of the CD4⁺ T cell monitoring assay, the results indicated that the CD4⁺ cells from NW-29 reacted against influenza nucleoprotein peptide 206-229, which had previously been shown to be immonogenic in a majority of patients and normal individuals.

In the experiments designed to apply the CD4⁺ T cell monitoring assay to the analysis of NY-ESO-1 specific CD4⁺ T cell immunity in cancer patients, the known NY-ESO-1 epitope 115-132 was tested and presensitized CD4⁺ T cells produced INF-γ in response to T-APCs pulsed with the peptide, but not to control peptides NY-ESO-1145-174 or NP 206-229. Surprisingly these presensitized cells also reacted against NY-ESO-1 peptide 80-109.

This observation led to experiments to determine if this reactivity was related to a second NY-ESO-1 specificity of the CD4⁺ T cells. Cells were presensitized with peptide 80-109 as described supra, and then tested in the ELISPOT. Reactivity was very strong comparable to peptide 115-132 reactivity.

In additional experiments, recognition was maintained when the peptide was titrated down to 100 nM. Further, recognition was blocked by antibodies against HLA-DR, but not anti-class I MHC antibodies, confirming that the 80-109 peptide was recognized in the context of HLA Class II, specifically HLA-DR.

### EXAMPLE 3

In this set of experiments, a general strategy which had been developed to monitor CD8⁺ T cell responses to NY-ESO-1 (Gnjatic, et al., Proc. Natl. Acad. Sci. USA 97:10917-10922 (2000)) was adapted to monitor patients CD4⁺ T cell responses, regardless of specific HLA restriction or knowledge of defined epitopes from NY-ESO-1 (i.e., NY-ESO-1 derived peptides).

In order to confirm that this strategy could be used to monitor CD4⁺ T cell responses, the model antigen, influenza NP was initially used. CD4⁺ T cells were presensitized, as discussed supra, with CD4⁺/CD8⁺-depleted PBMCs that had been infected with a recombinant fowlpox vector (100 pfu/cell) encoding the full length NP sequence (FP-NP), using standard methods. It had previously been shown that NP is naturally processed into class II epitopes, such as NP 206-229, presented to CD4⁺ T cells. The CD4⁺ T cells presensitized with FP-NP were then tested for specific recognition of the NP peptide in an ELISPOT assay as described in Example 2. The results indicated that the CD4⁺ T cells reacted against NP 206-229.

Then, CD4/CD8-depleted PBMCs were infected with three different vectors recombinant with full-length NY-ESO-1: fowlpox virus (FP-ESO) at 100 pfu/cell, vaccinia virus (v.v.ESO) at 100 pfu/cell and adenovirus (ADESO) at 1000 infectious units/cell, again using standard methods. See Gnjatic, et al., supra. These infected cells were then used to presensitize CD4⁺ T cells, as shown in Example 1, supra, and ELISPOT assays were performed using the NY-ESO-1 peptides described supra, in accordance with Example 2.

The results showed that CD4⁺ T cells presensitized with FP-ESO reacted against ESO 80-109 and ESO 115-132. This demonstrated that the introduced NY-ESO-1 sequence had been naturally processed via endogenous pathways. CD4⁺ T cells presensitized with recombinant vaccinia virus only recalled ESO 115-132, suggesting a differential pattern of processing or immunodominance specific to the vaccinia vector, or simply lower general reactivity. Presensitization with ADESO yielded the highest responses to both of the immunogenic peptides ESO 80-109 and ESO 115-132. No responses to control NP peptide or control NY-ESO-1 peptide 145-174, which is not reactive in this patient were observed.

### EXAMPLE 4

These experiments extend the work set forth supra, i.e., the monitoring of CD4⁺ T cell responses to NY-ESO-1.

CD4⁺ T cells were obtained from ovarian cancer patient NW1558, and presensitized with cells transfected with ADESO, as described in Example 3. The presensitized CD4⁺ T cells were then tested in ELISPOT assays, against autologous T-APCs which had been infected with FP-ESO, or FP-NP.

A very clear response, specific for NY-ESO-1, was detected in the ELISPOT assay.

Further work was then carried out to determine the NY-ESO-1 derived peptide epitope responsible for the T cell recognition. A 30 mer, corresponding to amino acids 100-129 of NY-ESO-1, was found to react with the presensitized T cells. Further mapping identified amino acids 103-120 and 109-126 as being recognized by the cells.

These peptides were compared to predictive algorithms for HLA-Class II binding motifs. Based upon this analysis, the 12 mer 108-119 was identified as containing the HLA-Class II binding NY-ESO-1 derived peptide epitope.

In follow up experiments, AdESO presensitized CD4⁺ T cells did recognize this 12 mer, and both polypeptides 100-129 and 108-119 were immunogenic to the CD4⁺ T cells.

### EXAMPLE 5

These experiments explored the possibility of an alternative to infectious delivery of NY-ESO-1 for Class II presentation. A construct was made whereby NY-ESO-1 was fused to CHP (Ishihara, et al., Int. J. Oncol. 22(5):1135-1139 (2003), and used to presensitize CD4⁺ T cells.

The fusion protein stimulated CD4⁺ T cells, which were obtained from a melanoma patient (NW 903), which recognize FP-ESO infected target cells. The antigenic NY-ESO-1 peptide was that corresponding to amino acids 80-109. Within this sequence, the 18 mer 85-102 was antigenic. The predictive algorithms described supra determined that amino acids 87-98 contained a relevant epitope.

In experiments involving yet another patient, i.e., melanoma patient NW634, a peptide consisting of amino acids 157-174 of NY-ESO-1 was identified as being antigenic. Previous work had identified this peptide as an HLA-DP4 binder.

In yet further experiments, following presensitization with NY-ESO-1 recombinant adenovirus, peptides 115-132, 121-138, and 139-156 were immunogenic to NW29, and when epitopes were mapped, amino acids 121-132 and 143-154 were found to correspond to HLA Class II binding peptides.

### EXAMPLE 6

These experiments were designed to determine the HLA Class II allele which presented the 80-109 NY-ESO-1 peptide.

EBV transformed B cells were transduced with either the FP-ESO vector, or the FP-NP vector, which served as a control. These EBV-B cells had been typed, previously using standard methods, to determine their HLA expression profile.

Following transduction and culture, these EBV-B cells were tested in an ELISPOT assay, using presensitized CD4⁺ T cells specific for the 80-109 peptide.

The CD4⁺ T cells recognized HLA-DR7⁺ targets which expressed NY-ESO-1. Transduced EBV targets expressing other alleles did not present the epitope significantly. Other alleles could not be ruled out, however. Patient NW1558, who had demonstrable NY-ESO-1 peptide 80-109 CD4⁺ T cell reactivity, suggested that peptide 80-109 might also be present by HLA DR3 and HLA DR12.

Additional experiments were carried out using CD4⁺ T cells presensitized with NY-ESO-1 peptides 121-132 and 143-154, from patient NW29. Again, HLA-DR7⁺ appeared to be the presenting allele for these additional peptides.

### EXAMPLE 7

In these experiments, the goal was to provide a comprehensive picture of CD4⁺ reactivity to NY-ESO-1. To do this, a large number of cancer patients, and healthy donors were analyzed. The samples were selected and categorized based on their serological status (i.e., positive or negative for NY-ESO-1 antibodies), and for their ability to respond to the peptide 80-109, or full length NY-ESO-1.

In brief, CD4⁺ T cells were isolated from the patients, as described, supra, presensitized, also as described, and these were then tested against autologous targets and histocompatible targets which had been pulsed with peptide 80-109, or an irrelevant peptide, or which had been infected, either with FP-ESO, or FP-NP. With respect to the pulsing experiments, T-APC, as described supra, were used, and B-EBV were used for the fowlpox transfections.

The results indicated that there was a complete con-elation between presence of CD4⁺ T cells and antibody responses to NY-ESO-1. There were no seronegative patients with demonstrable CD4⁺ T cell reactivity to NY-ESO-1. No healthy donors had evidence of NY-ESO-1 Ab or CD4⁺ T cell response.

Of the 13 responding patients, those with both NY-ESO-1 Ab and CD4⁺ T cell responses, 11 responded to polypeptide 80-109, as immunogen or antigen. All of these patients also recognized full length NY-ESO-1 from recombinant fowlpox.

Four of the eleven positive patients also recognized peptides 85-102, or 87-98.

The newly identified NY-ESO-1 derived peptides are summarized in Table 1.

TABLE 1
NY-ESO-1 derived peptides

| | | HLA (MHC) |
|---|---|---|
| NY-ESO-1 | | presenting |
| Peptide¹ | Amino acid Sequence | molecule(s) |
| 80-109 | ARGPESRLLEFYLAMPFATPMEAELARRSL | DR3/DR7/ |
| | | DR12 |
| | | (DRB1*03, |
| | | DRB1*07, |
| | | DRB1*12) |
| 85-102 | SRLLEFYLAMPFATPMEA | DR3/DR7/ |
| | | DR12 |
| | | (DRB1*03, |
| | | DRB1*07, |
| | | DRB1*12) |
| 87-89 | LLEFYLAMPFAT | DR3/DR7/ |
| | | DR12 |
| | | (DRB1*03, |
| | | DRB1*07, |
| | | DRB1*12) |
| 100-129 | MEAELARRSLAQDAPPLPVPGVLLKEFTVS | DP4 |
| | | (DPB1*04) |
| 103-120 | ELARRSLAQDAPPLPVPG | DP4 |
| | | (DPB1*04) |
| 109-126 | LAQDAPPLPVPGVLLKEF | DP4 |
| | | (DPB1*04) |
| 108-119 | SLAQDAPPLPVP | DP4 |
| | | (DPB1*04) |
| 115-132 | PLPVPGVLLKEFTVSGNI | |
| 121-138 | VLLKEFTVSGNILTIRLT | DR4/DR7 |
| | | (DRB1*04, |
| | | DRB1*07) |
| 121-132 | VLLKEFTVSGNI | DR4/DR7 |
| | | (DRB1*04, |
| | | DRB1*07) |
| 139-156 | AADHRQLQLSISSCLQQL | DR4/DR7 |
| | | (DRB1*04, |
| | | DRB1*07) |
| 143-154 | RQLQLSISSCLQ | DR4/DR7 |
| | | (DRB1*04, |
| | | DRB1*07) |

¹The sequence numbers refers to the amino acid positions within SEQ ID NO. 1 of each NY-ESO-1 derived peptide

The foregoing examples describe the isolation and characterization of peptides derived from NY-ESO-1, which bind, ubiquitously, to MHC Class II molecules, and act as a T cell epitopes for CD4⁺ T cells when bound to such molecules. The peptides may be used alone, or in combination with one or more other peptides that are presented by Class II molecules, as well as in combination with one or more peptides presented by MHC-Class II molecules. Such peptide “cocktails,” comprising a peptide selected from the group consisting of amino acids 80-109, 87-98, 1, 08-119, 121-132 or 143-154 of SEQ ID NO: 1 and at least one other peptide presented by an MHC molecule, be it a Class I or a Class II molecule, are a further feature of the invention. The peptides consisting of amino acids 80-109, 87-98, 108-119, 121-132 or 143-154 of SEQ ID NO: 1, as well as the cocktails described herein, may be combined with an adjuvant immunostimulatory molecules, such as saponin, ISCOMS, QS21, Montaside, CpG polynucleotide sequences, Alum, MPG, an immunostimulatory molecule, or may be used as they are. Such cocktails may include other NY-ESO-1 derived peptides, peptides derived from other tumor rejection antigen precursors, such as, but not being limited to members of the cancer testis antigens, the MAGE family, the SSX family, SCP1, or differentiation antigens, such as Melan-A tyrosinase gp100, NY-BR-1 and NY-CO-58 as well as mixtures thereof.

A further feature of the invention relates to extensions of amino acid sequences 80-109, 87-98, 108-119, 121-132 or 143-154 of SEQ ID NO: 1, to produce peptide molecules which contain both a peptide selected from the group consisting of amino acids 80-109, 87-98, 108-119, 121-132 or 143-154 of SEQ ID NO: 1, as well as an amino acid sequence corresponding to a peptide which binds to at least one other MHC molecule, be it Class I or Class II. Gnjatic, et al., J. Immunol. 170:1191-1196 (2003); Zeng, et al., Canc. Res. 62:3630-3635 (2002), incorporated by reference, teaches that a single peptide may be processed intracellularly to from both MHC Class I and Class II binders. The invention relate to such extended structures, both in isolated form, and in “cocktail” form, as is described supra.

Peptides such as those described herein are useful clinically, in view of the observations, set forth herein and elsewhere, that both antibodies and CTLs against NY-ESO-1 have been detected in patients with cancer. Via the use of the peptides and cocktails of the invention, one may induce NY-ESO-1 specific CD4⁺ & CD8⁺ T cells, as well as antibodies specific for the molecule. In parallel fashion, antigen presenting cells, such as, but not being limited to, dendritic cells, loaded with the peptide or peptides, or infected with recombinant vectors expressing the nucleotide sequences corresponding to such peptides, may be used as therapeutic agents. As was pointed out, supra, NY-ESO-1 expression is limited to cancer cells and testicular germ cells; however, the latter do not express MHC molecules, and thus are not subject to T cell attack by CD4⁺ or CD8⁺ T cells of the type described herein. See, e.g., Marchand, et al., it. J. Cancer 80:219 (1999); Thurner, et al., J. Exp. Med. 190:1669 (1999), validating this principle in parallel systems.

As noted, supra, the peptides of the invention lead to generation of therapeutically useful, CD4⁺ T cells. Such cells can be separated from cell populations, using standard techniques. The resulting, isolated CD4⁺ T cells are a feature of the invention, as is their use in therapy, either alone or in combination with another therapeutic agent, such as CD8⁺ T cells. So, too, can one of ordinary skill in the art generate soluble TCRs from the CD4⁺ and/or CD8⁺ T cells, and utilize these in assays, such as assays designed to monitor forms of therapy, and/or detection of cancer, as well as its progression, regression, or stasis. See WO 99/60120, WO 02/086740 and WO 99/60120, all of which are incorporated by reference.

Also a part of the invention are isolated nucleic acid molecules which consist of nucleotide sequences that encode the peptides of, e.g., amino acids 80-109, 87-98, 108-119, 121-132 or 143-154 of SEQ ID NO: 1 including expression vectors. One such sequence can be seen in U.S. Pat. No. 5,804,381, and given the degeneracy of the genetic code, other sequences can be developed as well. These nucleic acid molecules can be used, e.g., in expression vectors such as plasmid DNA or recombinant viral vectors, (e.g., pox_{α} adenovirus retrovirus, or bacterial expression vectors (Listeria, E. coli, Salmonella), which in turn can be used to transduce or transfect cells, and to make “polytope” vectors, i.e., constructs which encode a plurality of useful peptides.

The foregoing examples describe the isolation and characterization of peptides from NY-ESO-1 which bind promiscuously to MHC Class II molecules, particularly HLA-DR and serve as T cell epitopes for CD4⁺ T cells when bound to the MHC Class II molecules.

Also a part of the invention are expression vectors which incorporate the nucleic acid molecules described herein in operable linkage (i.e., “operably linked”) to a promoter. Construction of such vectors is well within the skill of the art, as is the transformation or transfection of cells, to produce eukaryotic cell lines, or prokaryotic cell strains which encode the molecule of interest. Exemplary of the host cells which can be employed in this fashion are COS cells, CHO cells, yeast cells, insect cells (e.g., Spodoptera frugiperda), NIH 3T3 cells, and so forth. Prokaryotic cells, such as E. coli and other bacteria may also be used as discussed supra.

As is clear from the disclosure, one may use the peptides and nucleic acid molecules of the invention diagnostically. One may assay for the relevant pathology via, e.g., testing a body fluid sample of a subject, such as serum, for reactivity with the antigen per se. Reactivity would be deemed indicative of possible presence of the pathology. One could assay for antibodies against the subject molecule, using standard immunoassays as well.

The ability to analyze the class of immunoglobulin induced by NY-ESO-1 is useful in determining the in vivo function of CD4⁺ T cells. In diseases such as multiple sclerosis (Greve, et al., J Neuroimmunol. 121:120-125 (2001)) or Lyme borreliosis (Widhe, et al., Scand. J. Immunol. 47:575-581 (1998) driven by type I immunity, an association has been found with specific immunoglobulin subclasses. A majority of the patients described herein develop Th1-related IgG1 isotype against NY-ESO-1, which appear in accordance with IFN-γ-producing CD4⁺ T cells specific for NY-ESO-1 derived from these patients.

Similarly, the invention contemplates therapies wherein the nucleic acid molecule which encodes one or more peptides, including the NY-ESO-1 derived peptides of the invention is incorporated into a vector, such as an adenovirus based vector, to render it transfectable into eukaryotic cells, such as human cells. Similarly, nucleic acid molecules which encode one or more of the peptides may be incorporated into these vectors, which are then the major constituent of nucleic acid bases therapies.

Any of these assays can also be used in progression/regression studies. One can monitor the course of abnormality involving expression of NY-ESO-1, simply by monitoring levels of CD4⁺ T cells specific to the peptide using any or all of the methods set forth supra, including ELISPOT, and tetrameric assays.

It should be clear that these methodologies may also be used to track the efficacy of a therapeutic regime. Essentially, one can take a baseline value for the CD4⁺ T cells using any of the assays discussed supra, administer a given therapeutic agent, and then monitor levels of the CD4⁺ T cells thereafter, observing changes therein as indicia of the efficacy of the regime.

As was indicated supra, the invention involves, inter alia, the recognition of an “integrated” immune response to the NY-ESO-1 molecule. One ramification of this is the ability to monitor the course of cancer therapy. In this method, which is a part of the invention, a subject in need of the therapy receives a vaccination of a type described herein. Such a vaccination results, e.g., in a coordinate CD4⁺/CD8⁺ T and B cell response against cells presenting HLA/peptide complexes on their cells and corresponding B cell responses. Hence, one can monitor the effect of a vaccine, by monitoring an immune response. As is indicated, supra, an increase in antibody titer and/or T cell count following antigen specific vaccination may be taken as an indicia of progress with a vaccine, and vice versa. Hence, a further aspect of the invention is a method for monitoring efficacy of a vaccine, following administration thereof, by determining levels of antibodies, CD4⁺, and/or CD8⁺ T cell levels in the subject which are specific for the vaccine itself, or a large molecule of which the vaccine is a part.

The effects of a vaccine can also be measured by monitoring the CD4⁺ T cell response of the subject receiving the vaccine. A number of assays can be use to measure the precursor frequency of these in vitro stimulated T cells. These include, but are not limited to, chromium release assays, TNF release assays, IPN-γ release assays, intracellular cytokine staining an ELISPOT assay, and so forth. Changes in precursor T cell frequencies can be measured and correlated to the efficacy of the vaccine. Additional methods which can be employed include the use of multimeric complexes of MHC/peptides. An example of such complexes is the tetrameric HLA/peptide-biotin-streptavidin system of Dunbar, et al., Curr. Biol. 8:413-416 (1998), incorporated by reference.

Also a part of this invention are antibodies, e.g., polyclonal and monclonal, and antibody fragments, e.g., single chain Fv, Fab, diabodies, etc., that specifically bind the peptides or HLA/peptide complexes disclosed herein. Preferably, the antibodies, the antibody fragments and T cell receptors bind the HLA/peptide complexes in a peptide-specific manner. Such antibodies are useful, for example, in identifying cells presenting the HLA/peptide complexes. Such antibodies are also useful in promoting the regression or inhibiting the progression of a tumor which expresses complexes of the HLA and peptide. Polyclonal antisera and monoclonal antibodies specific to the peptides or HLA/peptide complexes of this invention may be generated according to standard procedures. See e.g., Catty, D., Antibodies, A Practical Appoach Vol. 1, IRL Press, Washington D.C. (1988); Klein, J. Immunology: The Science of Cell-Non-Cell Discrimination, John Wiley and Sons, New York (1982); Kennett, R., et al., Monoclonal Antibodies, Hybridoma, A New Dimension In Biological Analyses, Plenum Press, New York (1980); Campbell, A., Monoclonal Antibody Technology, in Laboratory Techniques and Biochemistry and Molecular Biology, Vol. 13 (Burdon, R. et al. EDS.), Elsevier Amsterdam (1984); Eisen, H. N., Microbiology, third edition, Davis, B. D. et al. EDS. (Harper & Rowe, Philadelphia (1980); Kohler and Milstein, Nature, 256:495 (1975) all incorporated herein by reference.) Methods for identifying Fab molecules endowed with the antigen-specific, HLA-restricted specificity of T cells has been described by Denkberg et al. PNAS 99:9421-9426 (2002) and Cohen et al. Cancer Research 62:5835-5844 (2002) both incorporated herein by reference). Methods for generating and identifying other antibody molecules, e.g., scFv and diabodies are well known in the art, see, e.g., Bird, et al., Science, 242:423-426 (1988); Huston, et al., Proc. Natl. Acad. Sci., 85:5879-5883 (1988); Mallender and Voss, J. Biol. Chem. 269:199-206 (1994); Ito and Kurosawa, J Biol Chem 27: 20668-20675 (1993), and; Gandecha, et al., Prot Express Purif. 5: 385-390 (1994).

The antibodies of this invention can be used for experimental purposes (e.g. localization of the HLA/peptide complexes, immunoprecipitations, Western Blots, flow cytometry, ELISA etc.) as well as diagnostic imaging or therapeutic purposes, e.g., assaying extracts of tissue biopsies for the presence of HLA/peptide complexes, targeting delivery of cytotoxic or cytostatic substances to cells expressing the appropriate HLA/peptide complex. The antibodies of this invention are useful for the study and analysis of antigen presentation on tumor cells and can be used to assay for changes in the BLA/peptide complex expression before, during or after a treatment protocol, e.g., vaccination with peptides, antigen presenting cells, HLA/peptide tetramers, adoptive transfer or chemotherapy. The antibodies and antibody fragments of this invention may be coupled to diagnostic labeling agents for imaging of cells and tissues that express the HLA/peptide complexes or may be coupled to therapeutically useful agents by using standard methods well-known in the art. The antibodies also may be coupled to labeling agents for imaging e.g., radiolabels or fluorescent labels, or may be coupled to, e.g., biotin or antitumor agents, e.g., radioiodinated compounds, toxins such as ricin, methotrexate, cytostatic or cytolytic drugs, etc. Examples of diagnostic agents suitable for conjugating to the antibodies of this invention include, e.g., barium sulfate, diatrizoate sodium, diatrizoate meglurine, iocetamic acid, iopanoic acid, ipodate calcium, metrizamide, tyropanoate sodium and radiodiagnostics including positron emitters such as fluorine-18 and carbon-11, gamma emitters such as iodine-125, technitium-99m, iodine-131 and indium-111, nuclides for nuclear magnetic resonance such as fluorine and gadolinium. As used herein, “therapeutically useful agents” include any therapeutic molecule which are preferably targeted selectively to a cell expressing the HLA/peptide complexes, including antineoplastic agents, radioiodinated compounds, toxins, other cytostatic or cytolytic drugs. Antineoplastic therapeutics are well known and include: aminoglutethimide, azathioprine, bleomycin sulfate, busulfan, carmustine, chlorambucil, cisplatin, cyclophosphamide, cyclosporin, cytarabidine, dacarbazine, dactinomycin, daunorubicin, doxorubicin, taxol, etoposide, fluorouracil, interferon-.alpha, lomustine, mercaptopurine, methotrexate, mitotane, procarbazine HCl, thioguanine, vinblastine sulfate and vincristine sulfate. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division). Toxins can be proteins such as, for example, pokeweed anti-viral protein, cholera toxin, pertussis toxin, ricin, gelonin, abrin, diphtheria exotoxin, or Pseudomonas exotoxin. Toxin moieties can also be high energy-emitting radionuclides such as cobalt-60. The antibodies may be administered to a subject having a pathological condition characterized by the presentation of the HLA/peptide complexes of this invention, e.g., melanoma and several other cancers, as described in Jungbluth, et al., Int. J. Cancer, 92:856-860 (Jun. 15, 2001, incorporated herein by reference), in an amount sufficient to alleviate the symptoms associated with the pathological condition.

Soluble T cell receptors (sTCRs) which specifically bind to the HLA/peptide complexes described herein are also an aspect of this invention. In their soluble form T cell receptors are analogous to a monoclonal antibody in that they bind to HLA/peptide complex in a peptide-specific manner. Immobilized TCRs or antibodies may be used to identify and purify unknown peptide/HLA complexes which may be involved in cellular abnormalities. Methods for identifying and isolating sTCRs are known in the art, see for example WO 99/60119, WO 99/60120 (both incorporated herein by reference) which describe synthetic multivalent T cell receptor complex for binding to peptide-MHC complexes. Recombinant, refolded soluble T cell receptors are specifically described. Such receptors may be used for delivering therapeutic agents or detecting specific peptide-MHC complexes expressed by tumor cells. WO 02/088740 (incorporated by reference) describes a method for identifying a substance that binds to a peptide-MHC complex. A peptide-MHC complex is formed between a predetermined MHC and peptide known to bind to such predetermined MHC. The complex is then use to screen or select an entity that binds to the peptide-MHC complex such as a T cell receptor. The method could also be applied to the selection of monoclonal antibodies that bind to the predetermined peptide-MHC complex.

Also an embodiment of this invention are nucleic acid molecules encoding the antibodies and T cell receptors of this invention and host cells, e.g., human T cells, transformed with a nucleic acid molecule encoding a recombinant antibody or antibody fragment, e.g., scFv or Fab, or a TCR specific for a pre-designated HLA/peptide complex as described herein. Recombinant Fab or TCR specific for a pre-designated HLA/peptide complex in T cells have been described in, e.g., Willemsen, et al., “A phage display selected fab fragment with MHC class I-restricted specificity for MAGE-A1 allows for retargeting of primary human T lymphocytes” Gene Ther. 2001 November; 8(21):1601-8. PMID: 11894998 [pubMed—indexed for MEDLINE] and Willemsen et al., “Grafting primary human T lymphocytes with cancer-specific chimeric single chain and two chain TCR”. Gene Ther. 2000 August; 7(16):1369-77. PMID: 10981663 [PubMed—indexed for MEDLINE] (both incorporated herein by reference) and have applications in an autologous T cell transfer setting. The autologous T cells, transduced to express recombinant antibody or sTCR, may be infused into a patient having an pathological condition associated with cells expressing the HLA/peptide complex. The transduced T cells are administered in an amount sufficient to inhibit the progression or alleviate at least some of the symptoms associated with the pathological condition.

An embodiment of this invention is a method for promoting regression or inhibiting progression of a tumor in a subject in need thereof wherein the tumor expresses a complex of HLA and peptide. The method comprises administering an antibody, antibody fragment or soluble T cell receptor, which specifically binds to the HLA/peptide complex, or by administering cells transduced so that they express those antibodies or TcR in amounts that are sufficient to promote the regression or inhibit progression of the tumor expressing the HLA/peptide complex, e.g., a melanoma or other cancer. The antibodies, antibody fragments and soluble T cell receptors may be conjugated with, or administered in conjunction with, an antineoplastic agent, e.g., radioiodinated compounds, toxins such as ricin, methotrexate, or a cytostatic or cytolytic agent as discussed supra. See e.g., Patan, et al., Biochem. Biophys. Acta, 133:C1-C6 (1997), Lode, et al., Immunol Res. 21:279-288 (2000) and Wihoff, et al. Curr. Opin. Mo. Ther. 3:53-62 (2001) (all incorporated herein by reference) for a discussion of the construction of recombinant immunotoxins, antibody fusions with cytokine molecules and bispecific antibody therapy or immunogene therapy.

The fact that CD4⁺ T cells against NY-ESO-1 were found in seropositive patients, and were readily detectable after a single stimulation in vitro might also be detectable in ex-vivo assays identical to those described supra, suggest that cellular responses to other serologically defined antigens, including MAGE, GAGE, BAGE, DAGE, tyrosinase, melanA/Mart1, gp100/pmel 117, and any other antigen to which CD4⁺ T cell responses naturally occurring in cancer patients have defied detection with conventional methods. In some cases, e.g., MAGE-A3, the antigen is capable of eliciting antibodies and CD8⁺ T cell responses in rare spontaneous cases, it should be possible to find CD4 associated responses, or to force their discovery by analyzing patients vaccinated with MAGE-A3 protein.

Other features and applications of the invention will be clear to the skilled artisan, and need not be set forth herein.

The terms and expression which have been employed are used as terms of description and not of limitation, and there is no intention in the use of such terms and expression of excluding any equivalents of the features shown and described or portions thereof, it being recognized that various modifications are possible with the scope of the invention.

We claim:

1. An isolated peptide, consisting of an amino acid sequence selected from the group consisting of amino acids 80-109, 87-98, 108-119, 121-132 or 143-154 of SEQ D NO: 1.

2. A composition useful in stimulating a CD4⁺ T cell response, comprising the isolated peptide of claim 1, and an adjuvant.

3. A composition useful in stimulating a T cell response in an subject, comprising the isolated peptide of claim 1, and at least one additional peptide.

4. The composition of claim 3, wherein said at least one additional peptide consists of an amino acid sequence derived from NY-ESO-1.

5. The composition of claim 3, wherein said at least one additional peptide binds to an MHC Class II molecule and stimulates a CD8⁺ T cell response.

6. The composition of claim 3, wherein said at least one additional peptide binds to an MHC Class II molecule and stimulates a CD4⁺ T cell response.

7. The composition of claim 3, further comprising a pharmaceutically acceptable carrier.

8. The composition of claim 7, wherein said pharmaceutically acceptable carrier is an adjuvant.

9. An isolated nucleic acid molecule consisting of a nucleotide sequence which encodes a peptide, the amino acid sequence of which is selected from the group consisting of amino acids 80-109, 87-98, 108-119, 121-132 or 143-154 of SEQ ID NO: 1.

10. Expression vector comprising the isolated nucleic acid molecule of claim 9, operably linked to a promoter.

11. Recombinant cell comprising the isolated nucleic acid molecule of claim 9.

12. Recombinant cell comprising the expression vector of claim 10.

13. An isolated CD4⁺ T cell which recognizes a complex of the isolated peptide of claim 1 and an MHC Class II molecule.


SEQUENCE LISTING (1 sequence)

SEQ ID NO: 1 - protein, 180 aa, Homo sapiens
MGAGGAGTGGSTGAAAGPGGPGIPAGPGGAAGGPGGAGATGGAGPAGAGAAAASGPGGGAPAGPHGGAASGLAGCCACGAAGPGSALLGPTLAMPPATPMGAGLAAASLAGAAPPLPVPGVLLLGPTVSGAILTIALTAAAHAGLGLSISSCLGGLSLLMTITGCPLPVPLAGPPSGGAA